(19) United States
(12) Patent Application Publication
(10) Pub. No.: US 2003/0054385 A1
(43) Pub. Date: Mar. 20, 2003

(54) Human ubiquitin-conjugating enzymes

(76) Inventors: Preeti G. Lal, Santa Clara, CA (US); Jennifer L. Jackson, Fremont, CA (US); Neil C. Corley, Castro Valley, CA (US)

Correspondence Address: INCYTE GENOMICS, INC., 3160 PORTER DRIVE, PALO ALTO, CA, 94304, US

(21) Appl. No.: 10/157,669

(22) Filed: May 28, 2002

Related U.S. Application Data
(63) Continuation-in-part of application No. 09/520,076, filed on Mar. 7, 2000.
Application No. 09/520,076 is a division of application No. 09/359,967, filed on Jul. 22, 1999, now Pat. No. 6,146,624.
Application No. 09/359,967 is a division of application No. 08/965,689, filed on Nov. 6, 1997, now Pat. No. 6,015,702.
Application No. 08/965,689 is a continuation-in-part of application No. 08/933,750, filed on Sep. 23, 1997, now Pat. No. 5,932,442.

(51) Int. Cl. C12Q 1/68; C07H 21/04; C12N 9/64

(52) U.S. Cl. 435/6; 536/23.2; 435/226; 435/320.1; 435/325; 435/69.1

(57) ABSTRACT

The invention provides human ubiquitin-conjugating enzymes, cDNAs which encode the enzymes, and antibodies which specifically bind the enzymes. The invention also provides expression vectors, host cells, and antagonists and methods for diagnosing, treating or evaluating the treatment of disorders associated with differential expression of human ubiquitin-conjugating enzymes.

This application is a continuation-in-part application of co-pending U.S. Ser. No. 09/520,076, filed Mar. 07, 2000, which is a divisional of U.S. Pat. No. 6,146,624 which matured from U.S. Ser. No. 09/359,967, filed Jul. 22, 1999, which is a divisional of U.S. Pat. No. 6,015,702 which matured from U.S. Ser. No. 08/965,689, filed Nov. 06, 1997, which is a continuation-in-part of U.S. Pat. No. 5,932,442 which matured from U.S. Ser. No. 08/933,750, filed Sep. 23, 1997.

### FIELD OF THE INVENTION

This invention relates to human ubiquitin-conjugating enzymes, the cDNAs that encode those enzymes, and antibodies which specifically bind the enzymes and to the use of these compositions to diagnose, to stage, to treat, or to monitor the progression or treatment of neoplastic, immune, and neuronal disorders.

### BACKGROUND OF THE INVENTION

The ubiquitin conjugation system (UCS) plays a major role in the degradation of cellular proteins in eukaroytic cells and in some bacteria. The UCS mediates the elimination of abnormal proteins and regulates the half-lives of other important regulatory proteins that control gene transcription and cell cycle progression. The UCS is reported to degrade mitotic cyclic kinases, oncoproteins, tumor suppressors, viral proteins, transcriptional regulators, and receptors associated with signal transduction (Verma et al. (1997) Science 278:455-460; Ciechanover (1994) Cell 79:13-21).

There are several steps in the process of ubiquitin conjugation and protein degradation (Jentsch (1992) Annu Rev Genet 26:179-207). First, ubiquitin (Ub), a small, heat stable protein, is activated by a ubiquitin-activating enzyme (E1). This activation involves ATP-dependent binding of the C-terminus of Ub to the thiol group of an internal cysteine residue of E1. Then activated Ub is transferred to one of several Ub-conjugating enzymes (E2). Each E2 has a recognition subunit which allows it to interact with proteins carrying a particular degradation signal. E2 links the Ub molecule through its C-terminal glycine to an internal lysine of the target protein. It must be noted that different ubiquitin-dependent proteolytic pathways employ structurally similar, but distinct, E2s, and in some instances, accessory factors known as ubiquitin-ligases or E3s, are required to work in conjunction with E2s for recognition of certain substrates. More than one Ub molecule may be needed to ubiquinate a target protein which subsequently is recognized and degraded by a proteasome. After degradation, Ub is released and reutilized.

Prior to activation, Ub is usually expressed as a fusion protein composed of an N-terminal ubiquitin and a C-terminal extension protein (CEP) or as a polyubiquitin protein with Ub monomers attached head to tail. CEPs bear similarities to a variety of regulatory proteins in that most are highly basic, contain up to 30% lysine and arginine residues, and have nucleic acid-binding domains (Monia et al. (1989) J Biol Chem 264:4093-4103). The fusion protein is an important intermediate form which appears to allow co-regulation of the cell's translational and protein degradation activities and to localize inactive enzyme to specific cellular sites. Once delivered, C-terminal hydrolases cleave the fusion protein releasing Ub to carry out its work (Monia, supra).

E2s are important for substrate specificity in different UCS pathways. All E2s have a conserved UBC domain of approximately 16 kD and at least 35% identity and contain a centrally located cysteine residue which is required for ubiquitin-enzyme thiolester formation (Jentsch, supra). A highly conserved proline-rich element is located N-terminal to the active cysteine residue. Structural variations beyond the conserved domain are used to classify the E2 enzymes. The E2s of class 1 (E2-1) consist almost exclusively of the conserved UBC domain and include yeast E2-1 and UBCs 4, 5, and 7. These E2s a thought to require E3 to carry out their activities (Jentsch, supra). UBC7 has been shown to recognize ubiquitin as a substrate and to form polyubiquitin chains in vitro (van Nocker et al. (1996) J Biol Chem 271:12150-58). E2s of class II (E2-2) have various unrelated C-terminal extensions that contribute to substrate specificity and cellular localization. The yeast E2-2 enzymes, UBC2 and UBC3, have highly acidic C-terminal extensions that promote interactions with basic substrates such as histones. Yeast UBC6 has a hydrophobic signal-anchor sequence that localizes the protein to the endoplasmic reticulum.

Defects or alterations in the normal activity of the UCS are associated with a number of diseases and disorders. These include increased ubiquitin-dependent proteolysis as associated with cachexia (Llovera et al. (1995) Int J Cancer 61:138-141), degradation of the tumor-suppressor protein, p53 (Ciechanover, supra), and neurodegeneration such as observed in Alzheimer's disease (Gregori et al. (1994) Biochem Biophys Res Commun 203:1731-1738). Since ubiquitin conjugation is a rate-limiting step in antigen presentation, the ubiquitin degradation pathway may also have a critical role in the immune response (Grant et al. (1995) J Immunol 155: 3750-3758).

The discovery of new ubiquitin-conjugating enzymes, the cDNAs encoding them, and antibodies which specifically bind them satisfies a need in the art by providing new compositions useful to diagnose, to stage, to treat, or to monitor the progression or treatment of neoplastic, immune, and neuronal disorders.

### SUMMARY OF THE INVENTION

The invention is based on the discovery of ubiquitin conjugating enzymes that have been collectively designated as HUBI and individually, as HUBI-1 and HUBI-2, their encoding cDNAs, and antibodies which specifically bind the enzymes which are useful to diagnose, to stage, to treat, or to monitor the progression or treatment of neoplastic, immune, and neuronal disorders.

The invention provides an isolated cDNA comprising a polynucleotide encoding a protein having the amino acid sequence of SEQ ID NO:1 or the complement of the encoding cDNA. The invention also provides an isolated cDNA comprising a polynucleotide having the nucleic acid sequence of SEQ ID NO:2 or the complement of SEQ ID NO:2. The invention further provides probes comprising polynucleotides having the nucleic acid sequences of SEQ ID NOs:3-12, wherein the nucleic acid sequences have allelic homology to SEQ ID NO:2. The invention still further provides non-human homologs comprising polynucleotides having the nucleic acid sequences of SEQ ID NOs:13-16, wherein the nucleic acid sequences have at least 90% homology to SEQ ID NO:2.

The invention provides an isolated cDNA comprising a polynucleotide encoding a protein having the amino acid sequence of SEQ ID NO:17 or the complement of the encoding cDNA. The invention also provides an isolated cDNA comprising a polynucleotide having the nucleic acid sequence of SEQ ID NO:18 or the complement of SEQ ID NO:18. The invention further provides probes comprising polynucleotides having the nucleic acid sequences of SEQ ID NOs:19-27, wherein the nucleic acid sequences have allelic homology to SEQ ID NO:18. The invention still further provides non-human homologs comprising polynucleotides having the nucleic acid sequences of SEQ ID NOs:28-31, wherein the nucleic acid sequences have at least 91% homology to SEQ ID NO:18.

The invention provides a vector comprising the cDNA encoding HUBI, a host cell comprising the vector, and a method for using the cDNA to make the protein, the method comprising culturing the host cell comprising the vector comprising the cDNA encoding the protein under conditions for expression and recovering the protein from the host cell culture. The invention provides a purified protein. The invention also provides a transgenic cell line or organism comprising the vector comprising the cDNA encoding HUBI. The invention further provides a composition, a substrate or a probe comprising the cDNA, an allelic polynucleotide, or a non-human homolog which can be used in the methods of the invention. In one aspect, the probe is a single-stranded complementary RNA or DNA molecule.

The invention provides a method for using a cDNA, composition, a substrate, or a probe to detect the differential expression of a nucleic acid in a sample comprising hybridizing the cDNA, composition, a substrate, or probe to the nucleic acids, thereby forming hybridization complexes and comparing hybridization complex formation with a standard, wherein the comparison indicates the differential expression of the cDNA in the sample. In one aspect, the method further comprises amplifying the nucleic acids of the sample prior to hybridization. In another aspect, samples used in the method are from lung or bladder.

The invention also provides a method for using a cDNA to screen a library or plurality of molecules or compounds to identify at least one ligand which specifically binds the cDNA, the method comprising combining the cDNA with the molecules or compounds under conditions to allow specific binding and detecting specific binding to the cDNA, thereby identifying a ligand which specifically binds the cDNA. In one aspect, the molecules or compounds are selected from antisense molecules, artificial chromosome constructions, branched nucleic acids, DNA molecules, enhancers, peptides, peptide nucleic acids, proteins, RNA molecules, repressors, and transcription factors.

The invention provides a method for using a cDNA to purify a ligand which specifically binds the cDNA, the method comprising attaching the cDNA to a substrate, contacting the cDNA with a sample under conditions to allow specific binding, and dissociating the ligand from the cDNA, thereby obtaining purified ligand.

The invention provides a purified antibody which specifically binds the protein of the invention. The invention provides a method for using a protein to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein. In one aspect, the antibodies are selected from an intact immunoglobulin molecule, a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

The invention also provides methods for using a protein to prepare and purify polyclonal and monoclonal antibodies which specifically bind the protein. The method for preparing a polyclonal antibody comprises immunizing a animal with protein comprising a polypeptide having the amino acid sequence or SEQ ID NO:1 or SEQ ID NO:17 under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein comprising a polypeptide having the amino acid sequence or SEQ ID NO:1 or SEQ ID NO:17 under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention further provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the antibody is immobilized on a substrate. In another aspect, the amount of complex formation when compared to standards is diagnostic of cancer or complications of cancer, in particular lung or bladder cancer.

The invention still further provides a method for immunopurification of a protein comprising attaching an antibody to a substrate, exposing the antibody to a sample containing protein under conditions to allow antibody:protein complexes to form, dissociating the protein from the complex, and collecting purified protein. The invention also provides an array upon which HUBI, a cDNA encoding HUBI, or an antibody which specifically binds HUBI are immobilized.

The invention yet further provides a method for inserting a heterologous marker gene into the genomic DNA of a mammal to disrupt the expression of the endogenous polynucleotide and to produce a mammalian model system, the method comprising constructing a vector containing the cDNA selected from SEQ ID NOs:13-16 and 28-31, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, mammalian model system.

### BRIEF DESCRIPTION OF THE FIGURES

FIGS. 1A-1E show the amino acid sequence (SEQ ID NO:1) and nucleic acid sequence (SEQ ID NO:2) of human ubiquitin-conjugating enzyme, HUBI-1. The alignment was produced using MACDNASIS PRO software (Hitachi Software Engineering, South San Francisco Calif.).

FIGS. 2A-2D show the amino acid sequence (SEQ ID NO:17) and nucleic acid sequence (SEQ ID NO:18) of human ubiquitin-conjugating enzyme, HUBI-2. The alignment was produced using MACDNASIS PRO software (Hitachi Software Engineering).

FIG. 3 shows the amino acid sequence alignments among HUBI-1 (1762; SEQ ID NO:1), C. elegans (g1628097; SEQ ID NO:32), HUBI-2 (2456290; SEQ ID NO:17), and S. cerevisiae (g 4257; SEQ ID NO:33) produced using the MEGALIGN program of LASERGENE software (DNASTAR, Madison, Wis.).

### DESCRIPTION OF THE INVENTION

Before the proteins, nucleotide sequences, and methods are presented, it is to be understood that this invention is not limited to the particular methodology, protocols, cell lines, vectors, and reagents described, as these may vary. It is also to be understood that the terminology used is for the purpose of describing particular embodiments only, and is not intended to limit the scope of the present invention which will be limited only by the appended claims.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

The “complement” of a cDNA of the Sequence Listing refers to a nucleic acid molecule which is completely complementary over its full length and which will hybridize to a nucleic acid molecule under conditions of high stringency.

“cDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to conditions, diseases or syndromes in which HUBI and the cDNA encoding HUBI are differentially expressed; these include neoplastic, immune, and neuronal disorders and in particular, lung and bladder cancer.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses two-dimensional polyacrylamide electrophoresis (2D-PAGE, mass spectrophotometry (MS), enzyme-linked immunosorbent assays (ELISAs), radioimmunoassays (RIAs), and fluorescence activated cell sorting (FACS) or arrays and labeling moieties or antibodies to detect expression in a sample. The nucleic acids, proteins, or antibodies may be used in solution or immobilized on a substrate, and their detection is based on methods and labeling moieties well known in the art.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

HUBI refers to proteins having the amino acid sequences of ubiquitin-conjugating enzymes obtained from any species, particularly mammalian, including bovine, ovine, porcine, murine, equine, and preferably human, from any source whether natural, synthetic, semi-synthetic, or recombinant.

A “hybridization complex” is formed between a polynucleotide of the invention and a nucleic acid of a sample when the purines of one molecule hydrogen bond with the pyrimidines of the complementary molecule, eg, 5′-A-G-T-C-3′ base pairs with its complete complement, 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997, supra). BLAST2 may be used in a standardized and reproducible way to insert gaps in one of the sequences in order to optimize alignment and to achieve a more meaningful comparison between them. “Similarity” uses the same algorithms but takes conservative substitution of residues into account. In proteins, similarity exceeds identity in that substitution of a valine for a leucine or isoleucine, is counted in calculating the reported percentage. Substitutions which are considered to be conservative are well known in the art.

“Isolated or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels include but are not limited to anthocyanins, fluorescein, green fluorescent protein (GFP), β glucuronidase, lissamine, luciferase, phycoerythrin, rhodamine, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplicon, amplimer, primer, and oligomer.

A “pharmaceutical agent” may be an antisense molecule, a protein, a small drug molecule, a radionuclide, a cytotoxin such as vincristine, vinblastine, cisplatin, doxorubicin, methotrexate, and the like.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody.

“Sample” is used in its broadest sense as containing nucleic acids, proteins, and antibodies. A sample may comprise a bodily fluid such as ascites, blood, cerebrospinal fluid, lymph, saliva, semen, sputum, tears, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, tissue-print or tissue biopsy; buccal cells, skin, hair, a hair follicle; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The invention is based on the discovery of two new human ubiquitin conjugating enzymes (individually, HUBI-1 and HUBI-2 and collectively, HUBI), the cDNAs encoding HUBI, antibodies which specifically bind HUBI and to the use of these compositions to diagnose, to stage, to treat, or to monitor the progression or treatment of neoplastic, immune, and neuronal disorders. U.S. Pat. Nos. 5,932,442 and 6,015,702 are incorporated herein by reference in their entirety.

Nucleic acids encoding HUBI-1 of the present invention were first identified in Incyte Clone 1762 from the U937NOT01 cDNA library using a computer search for amino acid sequence alignments. SEQ ID NO:2 was derived from the extension and assembly of Incyte cDNAs 1762 (U937NOT01), 352606 (LYENNOT01), 1254927 (LUNGFET03), 1307911 (COLNFET02), 1359936 (LUNGNOT12), 1424618 (BEPINON01), 1503304 (BRAITUT07), 1833239 (BRAINON01), 2070865 (ISLTNOT01), and 2790509 (COLNTUT16) which SEQ ID NOs:3-12. The sequence of the reagent cDNA has been verified and is Incyte ID 5903277.

Transcript imaging presented in EXAMPLE V showed the very strong association of HUBI-1 with lung cancer. This analysis was followed by the microarray experiments. The table below shows the results of HUBI-1 expression across two experiments using microarrays and resected human primary lung tumor and matched microscopically normal lung tissue from the same donor. Differential expression (column 1) was considered significant if at least a two-fold difference (log₂=1.32) was observed. The first column shows the log2 value for the comparison, the second column, the description of the normal sample, the third column, the description of the cancer, and column 4, the donor number.

| log2(Cy5/Cy3) | Cy3 Sample | Cy5 Sample | Donor |
|---|---|---|---|
| 2.051567 | Normal Right Upper Lobe | AdenoCA, Right Upper Lobe | 7175 |
| 1.984427 | Normal Right Upper Lobe | AdenoCA, Right Upper Lobe | 7175 |

* AdenoCA = adenocarcinoma

The donor tissue for these experiments is described in EXAMPLE VI. These microarray experiments showed that HUBI-1 was differentially expressed in lung cancer. Therefore, when HUBI-1 or its encoding cDNA are used in a clinically relevant and tissue-specific manner with appropriate standards, their differential expression is diagnostic of lung cancer.

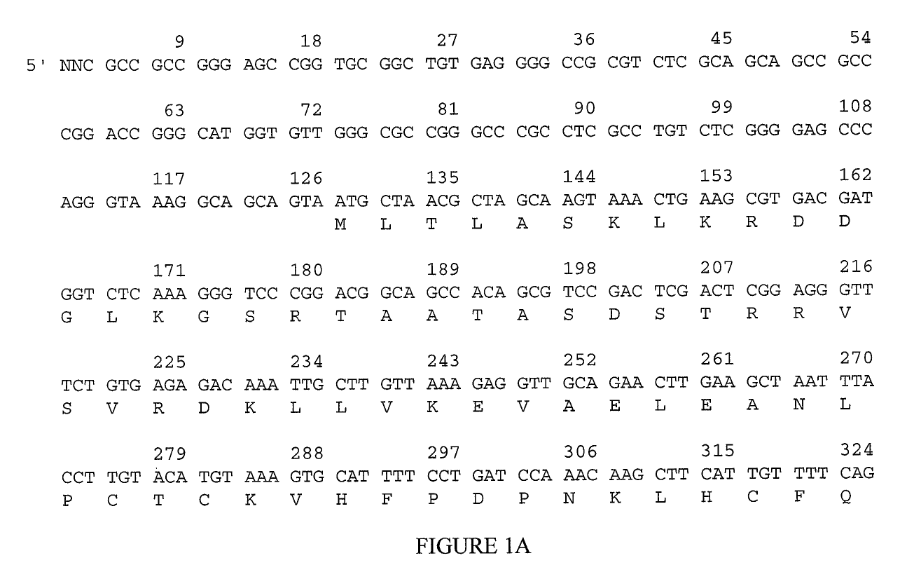

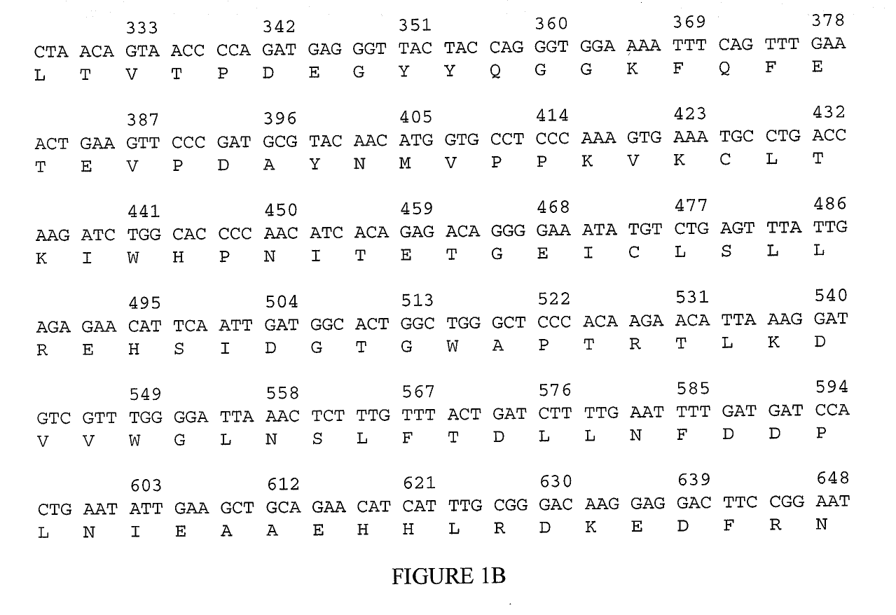

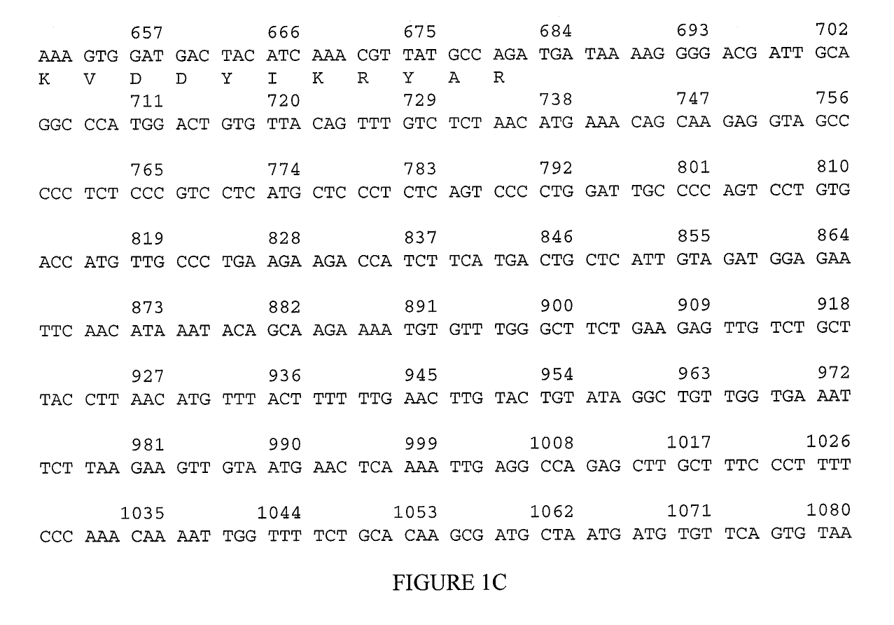

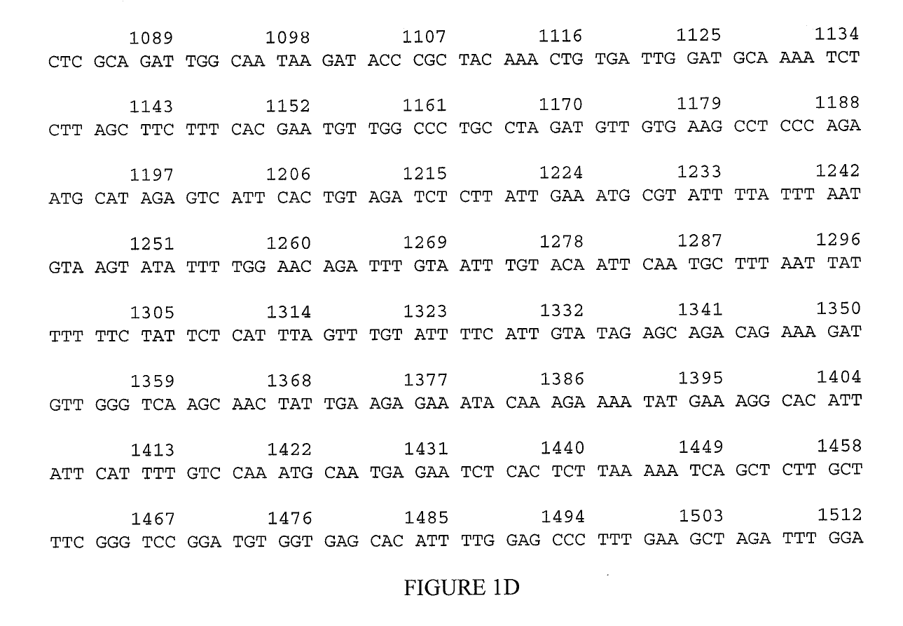

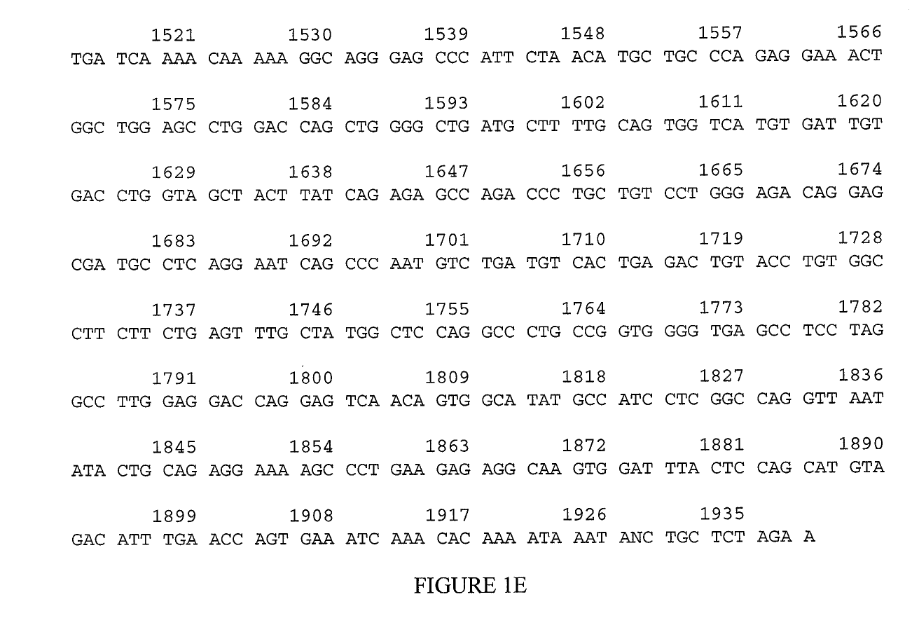

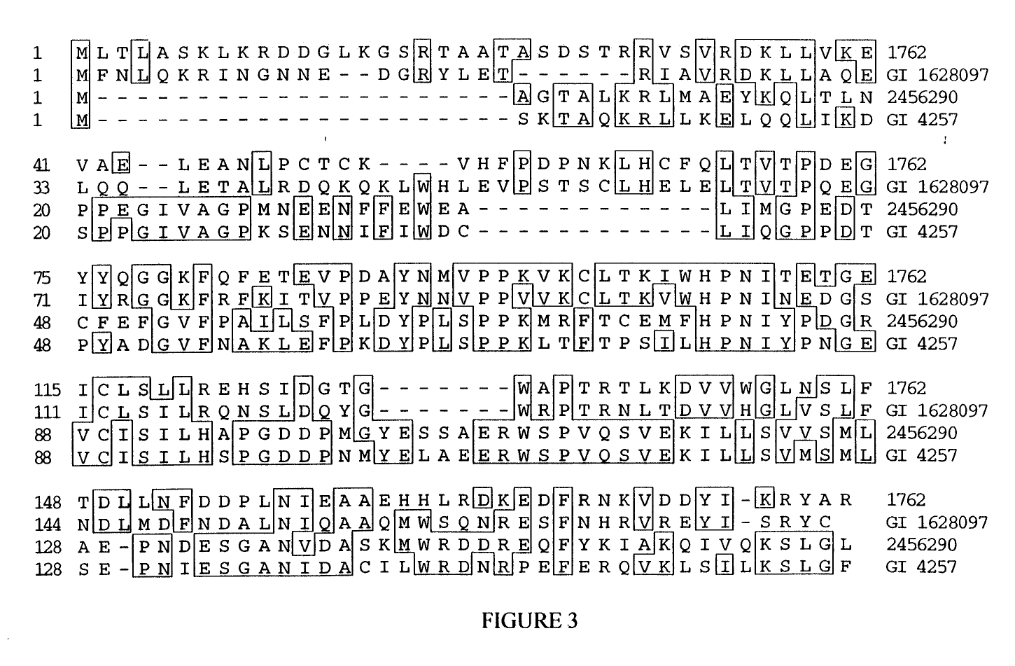

In one embodiment, the invention encompasses a polypeptide comprising the amino acid sequence of SEQ ID NO:1, as shown in FIGS. 1A-1E. HUBI-1 is 185 amino acids in length and has a potential N-glycosylation site at N₁₀₈; seven potential phosphorylation sites at T₂₂, S₂₆, T₂₇, S₃₁, T₅₁, T₇₀, and T₁₃₅; a ubiquitin conjugation motif at W₁₀₅HPNITETGEICLSL (SEQ ID NO:34); and a potential leucine zipper motif at L₁₃₆KDVVWGLNSLFTDLLNFDDPL (SEQ ID NO:35). As shown in FIG. 3, HUBI-1 has chemical and structural homology with a Caenorhabditis elegans protein (g1628097, SEQ ID NO:32). In particular, HUBI-1 and the C. elegans protein share 58% sequence identity, the phosphorylation sites at T₂₂, T₇₀, and T,₁₃₅, the ubiquitin conjugation motif, the conserved C₁₁₆ and the leucine zipper motif.

Nucleic acids encoding HUBI-2 of the present invention were first identified in Incyte Clone 2456290 from the ENDANOT01 cDNA library using a computer search for amino acid sequence alignments. SEQ ID NO:18 was derived from the extension and assembly of Incyte cDNAs: 728911 (LUNGNOT03), 1515858 (PANCTUT01), 1602091 (BLADNOT03), 1808143 (SINTNOT13), 2025691 (KERANOT02), 2122672 (BRSTNOT07), 2180113 (SININOT01), 2456290 (ENDANOT01), and 3406014 (ESOGNOT03) which are SEQ ID NOs:19-27. The sequence of the full length reagent cDNA has been verified and is Incyte ID 5669449CA2. The transcript image presented in EXAMPLE V showed the more than five-fold differential expression of HUBI-2 in bladder cancer. Therefore, when HUBI-2 or its encoding cDNA are used in a clinically relevant and tissue-specific manner with appropriate standards, their differential expression is diagnostic of bladder cancer.

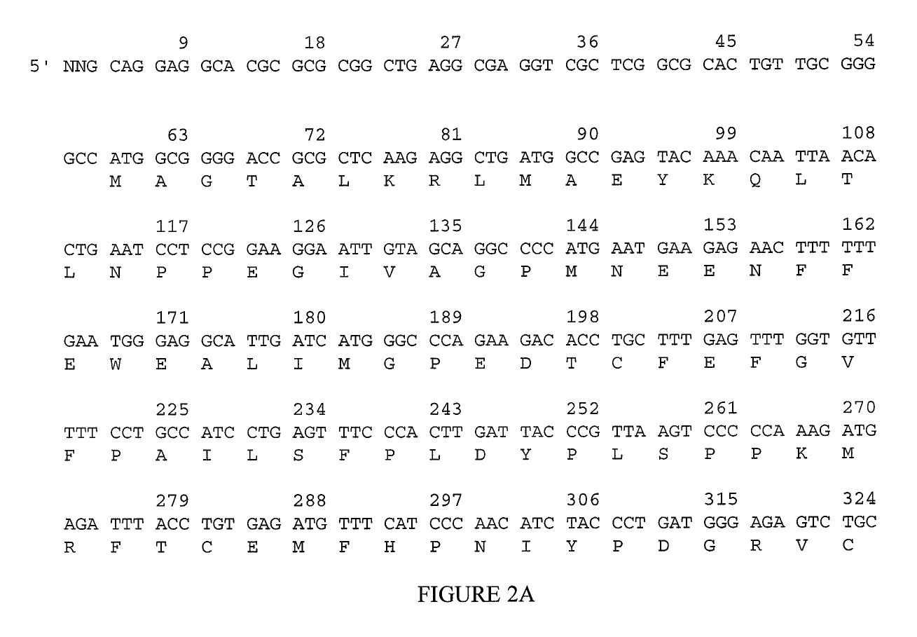

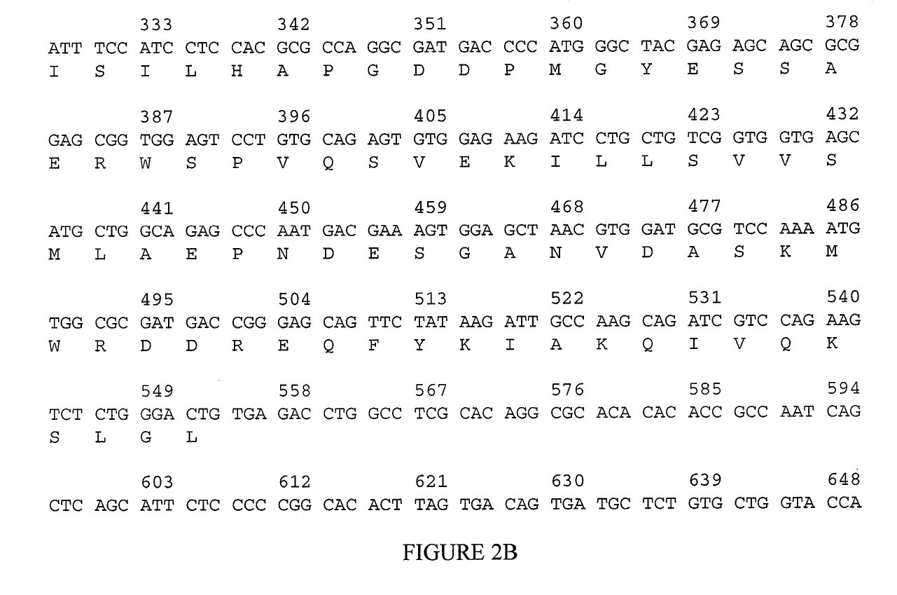

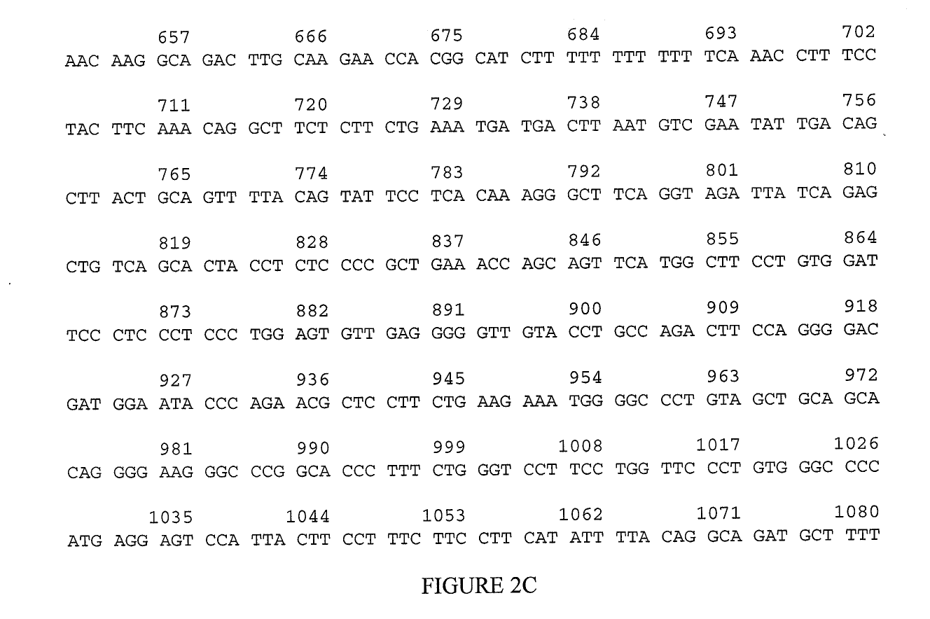

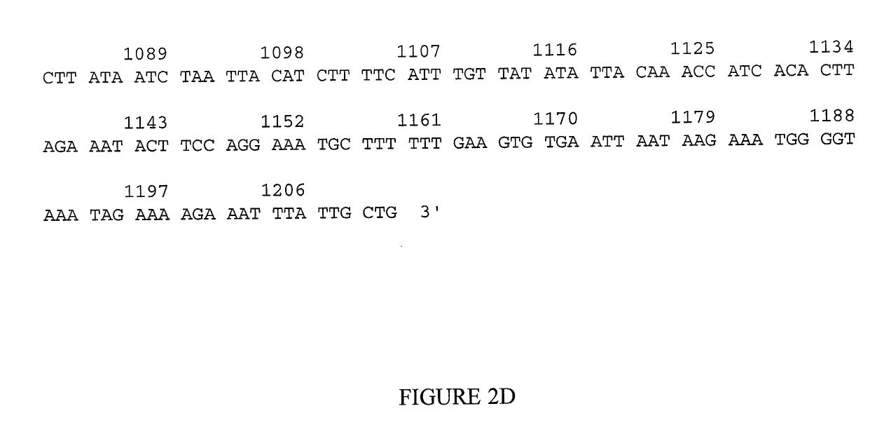

In one embodiment, the invention encompasses a polypeptide comprising the amino acid sequence of SEQ ID NO:17, as shown in FIGS. 2A-2D. HUBI-2 is 165 amino acids in length and has three potential phosphorylation sites at T₄₇, S₁₀₅, and Y₁₅₂ and a ubiquitin conjugation motif at F₇₈HPNIYPDGRVCISI (SEQ ID NO:36). As shown in FIG. 3, HUBI-2 has chemical and structural homology with a Saccharomyces cerevisiae ubiquitin conjugating enzyme (g4257, SEQ ID NO:33). In particular, HUBI-2 and the the S. cerevisiae protein share 62% sequence identity, the phosphorylation sites at T₄₇ and S_{105,} the ubiquitin conjugation motif, and the conserved C₈₉.

Mammalian homologs of the cDNAs encoding HUBI were identified using BLAST2 with default parameters and the ZOOSEQ databases (Incyte Genomics, Palo Alto Calif.). These highly homologous cDNAs average about 90% identity to all or part of the coding region of the human cDNA (H) as shown in the table below. The first column presents the SEQ ID NO of the nonhuman homolog (NH); the second column, the Incyte ID for the homologous cDNA; the third column, the species; the fourth column, the percent identity to the human cDNA; and the fifth column, the nucleotide alignment of the homolog cDNA to the human cDNA.

| | | | | | Nt_{H} |
|---|---|---|---|---|---|
| SEQ ID_{NH} | Incyte ID_{NH} | Species | % Identity | SEQ ID | Alignment |
| 13 | 273268_Rn.1 | Rat | 88% | 2 | 119-1422 |
| 14 | 023290_Mf.1 | Monkey | 98% | 2 | 641-1249 |
| 15 | 061683_Cf.1 | Dog | 85% | 2 | 903-1574 |
| 16 | 036419_Mm.3 | Mouse | 89% | 2 | 1-1384 |
| 28 | 222589_Rn.1 | Rat | 89% | 19 | 56-546 |
| 29 | 021278_Mf.1 | Monkey | 95% | 19 | 99-299 |
| 30 | 032543_Cf.1 | Dog | 90% | 19 | 407-733 |
| 31 | 035445_Mm.5 | Mouse | 89% | 19 | 56-546 |

It will be appreciated by those skilled in the art that as a result of the degeneracy of the genetic code, a multitude of cDNAs encoding HUBI, some bearing minimal similarity to the cDNAs of any known and naturally ocurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring HUBI, and all such variations are to be considered as being specifically disclosed.

The cDNAs of SEQ ID NOs:2-16 and 18-31 may be used in hybridization, amplification, and screening technologies to identify and distinguish among SEQ ID NO:2, SEQ ID NO:18, and related molecules in a sample. All of the mammalian cDNAs, but particularly SEQ ID NOs:13-16 and 28-31, may be used to produce transgenic cell lines or organisms which are model systems for human neoplastic, immune, and neuronal disorders and upon which the toxicity and efficacy of therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

Characterization and Use of the Invention

cDNA Libraries

In particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES. The consensus sequence is present in a single clone insert, or chemically assembled, based on the electronic assembly from sequenced fragments including Incyte cDNAs and extension and/or shotgun sequences. Computer programs, such as PHRAP (P Green, University of Washington, Seattle Wash.) and the AUTOASSEMBLER application (Applied Biosystems (ABI), Foster City Calif.), are used in sequence. After verification of the 5′ and 3′ sequence, at least one representative cDNA which encodes HUBI is designated a reagent for research and development.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Biosciences (APB), Piscataway N.J.), or combinations of polymerases and proofreading exonucleases (Invitrogen, San Diego Calif.). Sequence preparation is automated with machines such as the MICROLAB 2200 system (Hamilton, Reno Nev.) and the DNA ENGINE thermal cycler (MJ Research, Watertown Mass.) and sequencing, with the PRISM 3700, 377 or 373 DNA sequencing systems (ABI) or the MEGABACE 1000 DNA sequencing system (APB).

The nucleic acid sequences of the cDNAs presented in the Sequence Listing were prepared by such automated methods and may contain occasional sequencing errors and unidentified nucleotides (N) that reflect state-of-the-art technology at the time the cDNA was sequenced. Occasional sequencing errors and Ns may be resolved and SNPs verified either by resequencing the cDNA or using algorithms to compare multiple sequences; both of these techniques are well known to those skilled in the art who wish to practice the invention. The sequences may be analyzed using a variety of algorithms described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, can be removed, and deleted sequences can be restored to complete the assembled, finished sequences.

Extension of a Nucleic Acid Sequence

The sequences of the invention may be extended using various PCR-based methods known in the art. For example, the XL-PCR kit (ABI), nested primers, and cDNA or genomic DNA libraries may be used to extend the nucleic acid sequence. For all PCR-based methods, primers may be designed using software, such as OLIGO primer analysis software (Molecular Biology Insights, Cascade Colo.) to be about 22 to 30 nucleotides in length, to have a GC content of about 50% or more, and to anneal to a target molecule at temperatures from about 55 C to about 68 C. When extending a sequence to recover regulatory elements, genomic, rather than cDNA libraries are used.

Hybridization

The cDNA and fragments thereof can be used in hybridization technologies for various purposes. A probe may be designed or derived from unique regions such as the 5′ regulatory region or from a nonconserved region (ie, 5′ or 3′ of the nucleotides encoding the conserved catalytic domain of the protein) and used in protocols to identify naturally occurring molecules encoding the HUBI, allelic variants, or related molecules. The probe may be DNA or RNA, may be single-stranded, and should have at least 50% sequence identity to any of the nucleic acid sequences, SEQ ID NOs:2-16 and 18-31. Hybridization probes may be produced using oligolabeling, nick-translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using kits such as those provided by APB.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60 C, which permits the formation of a hybridization complex between nucleic acid sequences that contain some mismatches. Subsequent washes are performed at higher stringency with buffers such as 0.2×SSC with 0.1% SDS at either 45 C (medium stringency) or 68 C (high stringency). At high stringency, hybridization complexes will remain stable only where the nucleic acids are completely complementary. In some membrane-based hybridizations, from about 35% to about 50% formamide can be added to the hybridization solution to reduce the temperature at which hybridization is performed. Background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. (See, eg, U.S. Pat. No. 5,474,796; Schena et al. (1996) Proc Natl Acad Sci 93:10614-10619; Heller et al. (1997) Proc Natl Acad Sci 94:2150-2155; U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes, yeast artificial chromosomes, bacterial artificial chromosomes, bacterial P1 constructions, or the cDNAs of libraries made from single chromosomes.

QPCR

QPCR is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system (ABI) which consists of a 96-well thermal cycler connected to a laser and charge-coupled device (CCD) optics system. To perform QPCR, a PCR reaction is carried out in the presence of a doubly labeled probe. The probe, which is designed to anneal between the standard forward and reverse PCR primers, is labeled at the 5′ end by a fluorogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid, the sooner an increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T} values, preparing a standard curve, and determining starting copy number is performed using SEQUENCE DETECTOR 1.7 software (ABI).

Expression

Any one of a multitude of cDNAs encoding HUBI may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (sura).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors or plant cell systems transformed with expression vectors containing viral and/or bacterial elements (Ausubel supra, unit 16). In mammalian cell systems, an adenovirus transcription/translation complex may be utilized. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6×His, FLAG, MYC, and the like. GST and 6-His are purified using affinity matrices such as immobilized glutathione and metal-chelate resins, respectively. FLAG and MYC are purified using monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16).

Protein Identification

Several techniques have been developed which permit rapid identification of proteins using high performance liquid chromatography and mass spectrometry (MS). Beginning with a sample containing proteins, the method is: 1) proteins are separated using two-dimensional gel electrophoresis (2-DE), 2) selected proteins are excised from the gel and digested with a protease to produce a set of peptides; and 3) the peptides are subjected to mass spectral analysis to derive peptide ion mass and spectral pattern information. The MS information is used to identify the protein by comparing it with information in a protein database (Shevenko et al. (1996) Proc Natl Acad Sci 93:14440-14445). Proteins are separated by 2DE employing isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Probes, Eugene Oreg.) that is compatible with MS. Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a MALDI-TOF (Matrix Assisted Laser Desorption/Ionization-Time of Flight), ESI (Electrospray Ionization), and TOF-TOF (Time of Flight/Time of Flight) machines are used to determine a set of highly accurate peptide masses. Using analytical programs, such as TURBOSEQUEST software (Finnigan, San Jose Calif.), the MS data is compared against a database of theoretical MS data derived from known or predicted proteins. A minimum match of three peptide masses is used for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation (CID) to produce an ion series. The resulting fragmentation ions are analyzed in a second round of MS, and their spectral pattern may be used to determine a short stretch of amino acid sequence (Dancik et al. (1999) J Comput Biol 6:327-342). Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf. Creighton (1984) Proteins, Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative high performance liquid chromatography and its composition confirmed by amino acid analysis or by sequencing (Creighton, supra).

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich, St. Louis Mo.), and dinitrophenol may be used to increase immunological response. In humans, BCG (bacilli Calmette-Guerin) and Corynebacterium parvum increase response. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al. (1975) Nature 256:495-497; Kozbor et al. (1985) J Immunol Methods 81:31-42; Cote et al. (1983) Proc Natl Acad Sci 80:2026-2030; and Cole et al. (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are commonly used in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are discussed in Catty (supra) and Ausubel (supra) pp. 11.1-11.31.

Diagnostics

Labeling of Molecules for Assay

A wide variety of reporter molecules and conjugation techniques are known by those skilled in the art and may be used in various nucleic acid, amino acid, and antibody assays. Synthesis of labeled molecules may be achieved using kits such as those supplied by Promega (Madison Wis.) or APB for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes).

Nucleic Acid Assays

The cDNAs, fragments, oligonucleotides, complementary RNA and nucleic acid molecules, and peptide nucleic acids may be used to detect and quantify differential gene expression for diagnosis of a disorder. Similarly antibodies which specifically bind HUBI may be used to quantitate the protein. Disorders associated with such differential expression include neoplastic, immune, and neuronal disorders and particularly lung and bladder cancer. The diagnostic assay may use hybridization or amplification technology to compare gene expression in a biological sample from a patient to standard samples in order to detect differential gene expression. Qualitative or quantitative methods for this comparison are well known in the art.

Expression Profiles

A gene expression profile comprises the expression of a plurality of cDNAs as measured by after hybridization with a sample. The cDNAs of the invention may be used as elements on a array to produce a gene expression profile. In one embodiment, the array is used to diagnose or monitor the progression of disease. Researchers can assess and catalog the differences in gene expression between healthy and diseased tissues or cells.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is altered (higher or lower) in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose or stage that disorder.

By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disease, or disorder; or treatment of the condition, disease, or disorder. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and protein and antibody arrays. Such immunoassays typically involve the measurement of complex formation between the protein and its specific antibody. These assays and their quantitation against purifed, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. (See de Wildt et al. (2000) Nat Biotechnol

Differential expression of HUBI as detected using the cDNAs encoding HUBI, or an antibody that specifically binds HUBI and any of the above assays can be used to diagnose neoplastic, immune and neuronal disorders.

Therapeutics

As shown in FIG. 3 and described in THE INVENTION, chemical and structural similarity exists among HUBI and the protein sequences of C. elegans (g1628097) and S. cerevisiae (g4257). In addition, HUBI is differentially expressed in neoplastic, immune, and neuronal disorders in which HUBI plays a role in protein degradation. As shown in THE INVENTION and in EXAMPLES V, HUBI-1 is differentially expressed in lung cancer, and HUBI-2, in bladder cancer.

When decreased expression or activity of HUBI is desired, an inhibitor, antagonist, antibody or a pharmaceutical agent containing one or more of these molecules may be delivered. Such delivery may be effected by methods well known in the art and may include delivery by an antibody specifically targeted to the protein. In that degradation of tumor suppressor proteins such as p53 by E2 enzymes may contribute to the development of neoplastic disorders, HUBI expression may be decreased to ameliorate a neoplastic disorder. Such disorders may include, but are not limited to, adenocarcinoma, leukemia, lymphoma, melanoma, myeloma, sarcoma, and teratocarcinoma, and particularly cancers of the adrenal gland, bladder, bone, bone marrow, brain, breast, cervix, colon, esophagus, gall bladder, ganglia, heart, kidney, liver, lung, muscle, ovary, pancreas, parathyroid, penis, prostate, salivary glands, skin, small intestine, spleen, stomach, testis, thymus, thyroid, and uterus or complications of cancer such as cachexia. In one aspect, an antibody which specifically binds HUBI may be used directly as an antagonist or indirectly as a targeting or delivery mechanism for bringing a pharmaceutical agent to cells or tissue which express HUBI.

In addition, abnormalities in processing of neural proteins may contribute to neuronal disorders. Since HUBI appears to be involved in UCS dependent proteolysis and is found in neuronal tissues, HUBI expression may be decreased to ameliorate a neuronal disorder. Such disorders may include, but are not limited to, Alzheimer's disease, amyotrophic lateral sclerosis, bipolar disorder, Down's syndrome, epilepsy, Huntington's disease, multiple sclerosis, Parkinson's disease, and schizophrenia.

HUBI expression may also be decreased to ameliorate an immune disorder. Such disorders may include, but are not limited to, AIDS, adult respiratory distress syndrome, asthma, atherosclerosis, cholecystitis, Crohn's disease, ulcerative colitis, diabetes mellitus, emphysema, gastritus, glomerulonephritis, hypereosinophilia, irritable bowel syndrome, lupus erythematosus, multiple sclerosis, osteoarthritis, pancreatitis, and rheumatoid arthritis.

When increased expression or activity of the protein is desired, the protein, an agonist, or an enhancer may be delivered. Such delivery may be effected by methods well known in the art.

Any of the cDNAs, complementary molecules, or fragments thereof, proteins or portions thereof, vectors delivering these nucleic acid molecules or expressing the proteins, and their ligands may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or peptide nucleic acids) to the control, 5′, 3′, or other regulatory regions of the gene encoding HUBI. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of peptide nucleic acids and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule more resistant to endogenous endonucleases.

cDNA Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

Screening and Purification Assays

The cDNA encoding HUBI may be used to screen a library or a plurality of molecules or compounds for specific binding affinity. The libraries may be antisense molecules, artificial chromosome constructions, branched nucleic acids, DNA molecules, enhancers, peptides, peptide nucleic acids, proteins, RNA molecules, repressors, and transcription factors which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the cDNA.

In one embodiment, the cDNA of the invention may be incubated with a plurality of purified molecules or compounds and binding activity determined by methods well known in the art, eg, a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein to purify a ligand would involve combining the protein with a sample under conditions to allow specific binding, detecting specific binding between the protein and ligand, recovering the bound protein, and using a chaotropic agent to separate the protein from the purified ligand.

In a preferred embodiment, HUBI may be used to screen a plurality of molecules or compounds in any of a variety of screening assays. The portion of the protein employed in such screening may be free in solution, affixed to an abiotic or biotic substrate (eg, borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify agonists, antagonists, antibodies, DNA molecules, small drug molecules, immunoglobulins, inhibitors, mimetics, peptides, peptide nucleic acids, proteins, and RNA molecules or any other ligand, which specifically binds the protein.

In one aspect, this invention contemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity or therapeutic potential.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, gestation period, numbers of progeny, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to test whether long term administration may elicit toxicity, teratogenesis, or carcinogenesis. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, eg, U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the ability to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. Intone example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

### I cDNA Library Construction

U937NOT01

The U937NOT01 cDNA library, was constructed by Stratagene (STR937207), using RNA isolated from the U937 monocyte-like cell line. This cell line (ATCC CRL1593) was established by Sundstrom and Nilsson in 1974 (Int J Cancer 17:565-577) from malignant cells obtained from the pleural effusion of a 37-year-old Caucasian male with diffuse histiocytic lymphoma. cDNA synthesis was initiated using an XhoI-oligo d(T) primer. Double-stranded cDNA was blunted, ligated to EcoRI adaptors, digested with XhoI, size-selected, and cloned into the XhoI and EcoRI sites of the λ UNIZAP vector (Stratagene). The vector was transformed into E. coli host strain XL1-BLUE (Stratagene).

The cDNA library was screened with DNA probes, and the phagemids were obtained by the in vivo excision process, in which the host bacterial strain was coinfected with both the λ library phage and an f1 helper phage. Enzymes derived from both the library-containing phage and the helper phage nicked the λ DNA, initiated new DNA synthesis from defined sequences on the λ target DNA and created a smaller, single stranded circular DNA molecule that included the DNA sequence of the pBLUESCRIPT phagemid and the cDNA insert. The phagemid DNA was secreted from the cells, purified, and used to transform fresh host cells where double-stranded DNA was produced. The newly-transfected bacteria were selected on medium containing ampicillin.

ENDANOT01

The ENDANOT01 cDNA library was constructed from an aortic endothelial cell line derived from explanted heart/aorta tissue obtained from a male subject. The frozen cells were homogenized and lysed using a POLYTRON homogenizer (Brinkmann Instruments, Westbury N.Y.) in guanidinium isothiocyanate solution. The lysate was centrifuged over a 5.7 M CsCl cushion using an SW28 rotor in a L8-70M ultracentrifuge (Beckman Coulter, Fullerton Calif.) for 18 hours at 25,000 rpm at ambient temperature. The RNA was extracted with acid phenol, pH 4.7, precipitated using 0.3 M sodium acetate and 2.5 volumes of ethanol, resuspended in RNAse-free water, and treated with DNAse at 37 C. The RNA extraction and precipitation were repeated two times as before. The mRNA was isolated with the OLIGOTEX kit (Qiagen, Chatsworth Calif.) and used to construct the cDNA library.

The mRNA was handled according to the recommended protocols in the SUPERSCRIPT plasmid system (Invitrogen). The cDNAs were fractionated on a SEPHAROSE CL4B column (APB), and those cDNAs exceeding 400 bp were ligated into the pINCY plasmid. The plasmid was subsequently transformed into DH5∝ competent cells (Invitrogen).

### II Isolation and Sequencing of cDNAs

U937NOT01

Phagemid DNA was purified using the MAGIC MINIPREPS DNA purification system (Promega, Madison Wis.). The DNA was eluted from the purification resin already prepared for DNA sequencing and other analytical manipulations. Alternatively, phagemid DNA may be purified using the QIAWELL-8, QIAWELL PLUS and QIAWELL ULTRA DNA purification systems (Qiagen).

The cDNA inserts from random isolates of the U-937 library were sequenced in part. Methods for DNA sequencing are well known in the art. Conventional enzymatic methods employ Klenow fragment of DNA polymerase I, SEQUENASE, or Taq DNA polymerase (APB) to extend DNA chains from an oligonucleotide primer annealed to the DNA template of interest. Methods have been developed for the use of both single- and double-stranded templates. The chain termination reaction products were electrophoresed on urea-acrylamide gels and were detected either by autoradiography (for radionuclide-labeled precursors) or by fluorescence (for fluorescent-labeled precursors).

ENDANOT01

Plasmid DNA was released from the cells and purified using the REAL PREP 96 plasmid kit (Qiagen). The recommended protocol was employed except for the following changes: 1) the bacteria were inoculated into and cultured in 1 ml of sterile TERRIFIC BROTH (BD Biosciences, San Jose Calif.) with carbenicillin at 25 mg/L and glycerol at 0.4%; 2) the cultures were incubated for 19 hours, and the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4 C.

The cDNAs were prepared using a MICROLAB 2200 (Hamilton, Reno Nev.) in combination with Peltier thermal cyclers (MJ Research) and sequenced by the method of Sanger and Coulson (1975, J Mol Biol 94:441-448), and PRISM 377 DNA sequencing systems (ABI).

### III Extension of cDNAs

The cDNAs were extended using the cDNA clone and oligonucleotide primers. One primer was synthesized to initiate 5′ extension of the known fragment, and the other, to initiate 3′ extension of the known fragment. The initial primers were designed using primer analysis software to be about 22 to 30 nucleotides in length, to have a GC content of about 50% or more, and to anneal to the target sequence at temperatures of about 68 C to about 72 C. Any stretch of nucleotides that would result in hairpin structures and primer-primer dimerizations was avoided.

Selected cDNA libraries were used as templates to extend the sequence. If extension was performed than one time, additional or nested sets of primers were designed. Preferred libraries have been size-selected to include larger cDNAs and random primed to contain more sequences with 5′ or upstream regions of genes. Genomic libraries can be used to obtain regulatory elements extending into the 5′ promoter binding region.

High fidelity amplification was obtained by PCR using methods such as that taught in U.S. Pat. No. 5,932,451. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): The parameters for the cycles are 1: 94 C, three min; 2: 94 C, 15 sec; 3: 60 C, one min; 4: 68 C, two min; 5: 2, 3, and 4 repeated 20 times; 6: 68 C, five min; and 7: storage at 4 C. In the alternative, the parameters for primer pair T7 and SK+ (Stratagene) were as follows: 1: 94 C, three min; 2: 94 C, 15 sec; 3: 57 C, one min; 4: 68 C, two min; 5: 2, 3, and 4 repeated 20 times; 6: 68 C, five min; and 7: storage at 4 C.

The concentration of DNA in each well was determined by dispensing 100 μl PICOGREEN quantitation reagent (0.25% reagent in 1×TE, v/v; Molecular Probes) and 0.5 μl of undiluted PCR product into each well of an opaque fluorimeter plate (Corning Life Sciences, Acton Mass.) and allowing the DNA to bind to the reagent. The plate was scanned in a Fluoroskan II (Labsystems Oy, Helsinki Finland) to measure the fluorescence of the sample and to quantify the concentration of DNA. A 5 μl to 10 μl aliquot of the reaction mixture was analyzed by electrophoresis on a 1% agarose minigel to determine which reactions were successful in extending the sequence.

The extended clones were desalted, concentrated, transferred to 384-well plates, digested with CviJI cholera virus endonuclease (Molecular Biology Research, Madison Wis.), and sonicated or sheared prior to religation into pUC18 vector (APB). For shotgun sequences, the digested nucleotide sequences were separated on low concentration (0.6 to 0.8%) agarose gels, fragments were excised, and the agar was digested with AGARACE enzyme (Promega). Extended clones were religated using T4 DNA ligase (New England Biolabs) into pUC18 vector (APB), treated with Pfu DNA polymerase (Stratagene) to fill-in restriction site overhangs, and transfected into E. coli competent cells. Transformed cells were selected on antibiotic-containing media, and individual colonies were picked and cultured overnight at 37 C in 384-well plates in LB/2×carbenicillin liquid media.

The cells were lysed, and DNA was amplified using primers, Taq DNA polymerase (APB) and Pfu DNA polymerase (Stratagene) with the following parameters: 1: 94 C, three min; 2: 94 C, 15 sec; 3: 60 C, one min; 4: 72 C, two min; 5: 2, 3, and 4 repeated 29 times; 6: 72 C, five min; and 7: storage at 4 C. DNA was quantified using PICOGREEN quantitation reagent (Molecular Probes) as described above. Samples with low DNA recoveries were reamplified using the conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

### IV Homology Searching of cDNA Clones and Their Deduced Proteins

The nucleotide sequences of the Sequence Listing or amino acid sequences deduced from them were used as query sequences against databases such as GenBank, SwissProt, BLOCKS, and Pima II. These databases which contain previously identified and annotated sequences were searched for regions of similarity using BLAST.

BLAST produced alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith and Smith (1992; Protein Engineering 5:35-51), incorporated herein by reference, can be used when dealing with primary sequence patterns and secondary structure gap penalties. As disclosed in this application, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) and incorporated herein by reference, searches matches between a query sequence and a database sequence, to evaluate the statistical significance of any matches found, and to report only those matches which satisfy the user-selected threshold of significance. In this application, threshold is set at 10-25 for nucleotides and 10-14 for peptides.

### V Northern Analysis and Transcript Imaging

Northern Analysis

Northern analysis is a laboratory technique used to detect the presence of a transcript of a gene and involves the hybridization of a labeled nucleotide sequence to a membrane to which RNAs or cDNAs from a particular cell type or tissue have been immobilized (Sambrook, supra).

Analogous computer techniques using BLAST were used to search for identical or related molecules in nucleotide databases such as GenBank or the LIFESEQ databases (Incyte Genomics). This analysis is much faster than multiple, membrane-based hybridizations, and the sensitivity of the computer search can be modified. The basis of the search is the product score which is defined as:

% sequence identity×% maximum BLAST score/100

The product score takes into account both the degree of similarity between two sequences and the length of the sequence match. For example, with a product score of 40, the match will be exact within a 1-2% error; and at 70, the match will be exact. Homologous molecules are usually identified by selecting those which show product scores between 15 and 40, although lower scores may identify related molecules.

The results of northern analysis were reported as disease categories in which the transcript encoding HUBI occurs. Northern analysis of HUBI-1 showed expression in various cDNA libraries, 47% of the which are associated with immune response, 35% of which are associated with neoplastic disorders, and 17% of which are associated with development. Northern analysis of HUBI-2 showed expression in various cDNA libraries; 31% of which are associated with cancers; 22% of the which are associated with immune response, and 16% of which are associated with the nervous system.

Transcript Imaging

The transcript images below were performed using the LIFESEQ GOLD database (Incyte Genomics). This process allows assessment of the relative abundance of the expressed polynucleotides in all of the cDNA libraries and was described in U.S. Pat. No. 5,840,484, incorporated herein by reference. All sequences and cDNA libraries in the LIFESEQ Gold database are categorized by system, organ/tissue and cell type. The categories include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. Criteria for transcript imaging can be selected from category, number of cDNAs per library, library description, disease indication, clinical relevance of sample, and the like.

For each category, the number of libraries in which the sequence was expressed are counted and shown over the total number of libraries in that category. For each library, the number of cDNAs are counted and shown over the total number of cDNAs in that library. In some transcript images, all enriched, normalized or subtracted libraries, which have high copy number sequences can be removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs such as heart, kidney, nerves or pancreas would be aided by removing clinical samples from the analysis.

The transcript images for SEQ ID NOs:2 and 18 are shown in the tables below. The first column shows library name; the second column, the number of cDNAs sequenced in that library; the third column, the description of the library; the fourth column, absolute abundance (Abund) of the transcript in the library; and the fifth column, percentage abundance of the transcript in the library.

SEQ ID NO:2

Category: Respiratory System (Lung)

| | | | | % |
|---|---|---|---|---|
| Library | cDNAs | Description of Tissue | Abund | Abund |
| LUNGNOT12 | 3579 | lung, mw/adenocarcinoma, COPD, 78M | 2 | 0.0559 |
| LUNLTUT11 | 3859 | squamous cell carcinoma, 50M | 2 | 0.0518 |
| LUNPTUT02 | 3640 | mets uterine leiomyosarcoma, 55F | 1 | 0.0275 |
| LUNGTUT12 | 3736 | adenocarcinoma, 70F | 1 | 0.0268 |
| LUNGTUT07 | 3873 | squamous cell carcinoma, 50M | 1 | 0.0258 |
| LUNGTUP04 | 17658 | neuroendocrine carcinoid, pool, NORM, | 4 | 0.0227 |
| | | 3’ CGAP | | |
| LUNGTUP07 | 30384 | neuroendocrine carcinoid, pool, SUB, | 6 | 0.0197 |
| | | CGAP | | |
| LUNGFET03 | 10926 | lung, aw/anencephaly, fetal, 20wF | 1 | 0.0092 |

The expression of SEQ ID NO:2 was strongly associated with lung cancers and was not expressed in lung tissues from patients diagnosed with asthma (LUNGAST01, LUNGNOT33, LUNGNOT38, and LUNGNOT39), idiopathic pulmonary disease (LUNGDIN02, LUNGDIS03), pneumonitis (LUNGNOT15) or emphysema (LUNGNOT20). Nor was SEQ ID NO:2 expressed in cytologically normal lung samples (LUNGNOF03, LUNGNOM01, LUNGNON07, LUNGNOP01, LUNGNOP03, LUNGNOP04, LUNGNOT01, LUNGNOT02, LUNGNOT03, LUNGNOT04, LUNGNOT18, LUNGNOT22, LUNGNOT23, LUNGNOT25, LUNGNOT27, LUNGNOT28, LUNGNOT30, LUNGNOT31, LUNGNOT34, LUNGNOT35, LUNGNOT37, LUNGNOT40, LUNGTMC01, LUNGTMT03, and LUNGTMT04) or in fetal tissue samples (LUNGFEC01, LUNGFEM01, LUNGFEN02, LUNGFEP01, LUNGFEP02, LUNGFER04, LUNGFET04, LUNGFET05, LUNGNOT09, and LUNGNOT10).

SEQ ID NO:18

Category: Urinary System (Bladder)

| | | | | % |
|---|---|---|---|---|
| Library | cDNAs | Description of Tissue | Abund | Abund |
| BLADTUP01 | 1161 | bladder cancer, pool, LICR, EF | 2 | 0.1723 |
| BLADNOT03 | 3676 | bladder, mw/TC CA, 80F, | 1 | 0.0272 |
| | | m/BLADTUT02 | | |
| BLADNOT06 | 3731 | bladder, mw/TC CA, 66M, | 1 | 0.0268 |
| | | m/BLADTUT05 | | |
| BLADDIT01 | 3774 | bladder, chronic cystitis, aw/urethral | 1 | 0.0265 |
| | | adenocarcinoma, 73M | | |
| BLADNOT09 | 4111 | bladder, mw/TC CA, 58M, | 1 | 0.0243 |
| | | m/BLADTUT03 | | |

SEQ ID NO:18 was more than five-fold differentially expressed in cancer of the bladder. It was not significantly expressed in any cytologically normal bladder including BLADNOT03, BLADNOT06, BLADDIT01, and BLADNOT04 shown in the table above and BLADNOP01, BLADNOR01, BLADNOT01, BLADNOT04, BLADNOT05, and BLADNOT08 which had no expression. SEQ ID NO:18, when used in a clinically relevant, tissue-specific assay, is diagnostic for cancer of the bladder.

### VI Hybridization and Amplication Technologies and Analyses

Tissue Samples

Matched normal and cancerous lung tissue samples were provided by the Roy Castle International Centre for Lung Cancer Research (Liverpool UK). The description for Donor 7175 was moderately differentiated, adenocarcinoma, stage IB, and grossly uninvolved lung tissue were removed from a 67 year-old male donor. The tumor sample showed 50% overt tumor cells within the tumor and less than 5% overt tumor cells in the uninvolved tissue.

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 110 C oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (Tropix, Bedford Ma.) for 30 min at 60 C; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C for 10 min. The labeling reaction is stopped by adding 5 μl of 0.2M EDTA, and probe is purified from unincorporated nucleotides using a PROBEQUANT G-50 microcolumn (APB). The purified probe is heated to 100 C for five min, snap cooled for two min on ice, and used in membrane-based hybridizations as described below.

Probe Preparation for QPCR

Probes for the QPCR are prepared according to the ABI protocol.

Probe Preparation for Polymer Coated Slide Hybridization

The following method is used for the preparation of probes for the microarray analysis. Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1×yeast control mRNAs. Yeast control mRNAs are synthesized by in vitro transcription from noncoding yeast genomic DNA (W Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C for two hr. The reaction mixture is then incubated for 20 min at 85 C, and probes are purified using two successive CHROMA SPIN+TE 30 columns (Clontech, Palo Alto Calif.). Purified probe is ethanol-precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65 C for five min, and mixed thoroughly. The probe is heated, mixed as before, stored on ice and used in high density array-based hybridizations as described below.

Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55 C for two hr. The probe, diluted in 15 ml fresh hybridization solution, is then added to the membrane. The membrane is hybridized with the probe at 55 C for 16 hr. Following hybridization, the membrane is washed for 15 min at 25 C in 1 mM Tris (pH 8.0), 1% Sarkosyl, and four times for 15 min each at 25 C in 1 mM Tris (pH 8.0). To detect hybridization complexes, XOMAT-AR film (Eastman Kodak, Rochester N.Y.) is exposed to the membrane overnight at −70 C, developed, and examined visually.

Polymer Coated Slide-based Hybridization

The following method was used in the microarray analysis presented in Table 3. Probe is heated to 65 C for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C in 1×SSC, 0.1% SDS, and three times for 10 min each at 45 C in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20×microscope objective (Nikon, Melville N.Y.). The slide containing the array is placed on a computer-controled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1: 100,000.

The output of the photomultiplier tube is digitized using a 12-bit RTI-835H analog-to-digital (A/D) conversion board (Analog Devices, Norwood Ma.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

QPCR Analysis

For QPCR, cDNA is synthesized from 1 ug total RNA in a 25 ul reaction with 100 units M-MLV reverse transcriptase (Ambion, Austin Tex.), 0.5 mM dNTPs (Epicentre, Madison Wis.), and 40 ng/ml random hexamers (Fisher Scientific, Chicago Ill.). Reactions are incubated at 25 C for 10 minutes, 42 C for 50 minutes, and 70 C for 15 minutes, diluted to 500 ul, and stored at −30 C. Alternatively, cDNA is obtained from Human MTC panels (Clontech Laboratories, Palo Alto Calif.). PCR primers and probes (5′6-FAM-labeled, 3′TAMRA) are designed using PRIMER EXPRESS 1.5 software (ABI) and synthesized by Biosearch Technologies (Novato Calif.) or ABI.

QPCR reactions are performed using an PRISM 7700 detection system (ABI) in 25 ul total volume with 5 ul cDNA template, 1×TAQMAN UNIVERSAL PCR master mix (ABI), 100 nM each PCR primer, 200 nM probe, and 1×VIC-labeled beta-2-microglobulin endogenous control (ABI). Reactions are incubated at 50 C for 2 minutes, 95 C for 10 minutes, followed by 40 cycles of incubation at 95 C for 15 seconds and 60 C for 1 minute. Emissions are measured once every cycle, and results are analyzed using SEQUENCE DETECTOR 1.7 software (ABI) and fold differences, relative concentration of mRNA as compared to standards, are calculated using the comparative C_{T} method (ABI User Bulletin #2).

### VII Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in triple helix base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

### VIII Expression of HUBI

Expression of HUBI is accomplished by subcloning the cDNAs into appropriate vectors and transforming the vectors into host cells. In this case, the cloning vector is also used to express HUBI in E. coli. Upstream of the cloning site, this vector contains a promoter for β-galactosidase, followed by sequence containing the amino-terminal Met, and the subsequent seven residues of β-galactosidase. Immediately following these eight residues is a bacteriophage promoter useful for transcription and a linker containing a number of unique restriction sites.

Induction of an isolated, transformed bacterial strain with IPTG using standard methods produces a fusion protein which consists of the first eight residues of β-galactosidase, about 5 to 15 residues of linker, and the full length protein. The signal residues direct the secretion of HUBI into the bacterial growth media which can be used directly in the following assay for activity.

### IX Demonstration of HUBI Activity

HUBI activity is demonstrated by the formation of di-ubiquitin conjugates from free ubiquitin (van Nocker, supra). HUBI is incubated together with 75 pmol ¹²⁵I-labeled ubiquitin, 20 nM wheat E1, 2 mM Mg ATP, 0.1 mM dithiothreitol, and 50 mM Tris-HCl, pH 8.0. The reaction is incubated for 2 minutes at 4 C and the di-ubiquitin product separated from free ubiquitin by polyacrylamide gel electrophoresis. Di-ubiquitin is visualized by autoradiography, removed from the gel, and counted in a gamma radioisotope counter. The amount of di-ubiquitin formed in the reaction is proportional to the activity of HUBI in the assay.

### X Production of HUBI Specific Antibodies

HUBI that is purified using PAGE electrophoresis (Sambrook, supra), or other purification techniques, is used to immunize rabbits and to produce antibodies using standard protocols. The amino acid sequences of SEQ ID NO:1 and SEQ ID NO:17 are analyzed using LASERGENE software (DNASTAR) to determine regions of high immunogenicity and corresponding oligopeptides are synthesized and used to raise antibodies by means known to those of skill in the art. Selection of appropriate epitopes, such as those near the C-terminus or in hydrophilic regions, is described by Ausubel (supra).

Typically, the oligopeptides are 15 residues in length, synthesized using an 431A Peptide synthesizer (ABI) using fmoc-chemistry, and coupled to KLH (Sigma-Aldrich) by reaction with N-maleimidobenzoyl-N-hydroxysuccinimide ester (Ausubel, supra). Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radio-iodinated, goat anti-rabbit IgG.

### XI Purification of Naturally Occurring HUBI Using Specific Antibodies

Naturally occurring or recombinant HUBI is purified by immunoaffinity chromatography using antibodies specific for HUBI. An immunoaffinity column is constructed by covalently coupling HUBI antibody to an activated chromatographic resin, such as CNBr-activated SEPHAROSE (APB). After the coupling, the resin is blocked and washed according to the manufacturer's instructions.

Media containing HUBI is passed over the immunoaffinity column, and the column is washed under conditions that allow the preferential absorbance of HUBI (eg, high ionic strength buffers in the presence of detergent). The column is eluted under conditions that disrupt antibody/HUBI binding (eg, a buffer of pH 2-3 or a high concentration of a chaotrope, such as urea or thiocyanate ion), and HUBI is collected.

### XII Identification of Molecules Which Interact with HUBI

HUBI or biologically active fragments thereof are labeled with ¹²⁵I Bolton-Hunter reagent (Bolton et al. (1973) Biochem J 133:529-39). Candidate molecules previously arrayed in the wells of a multi-well plate are incubated with the labeled HUBI, washed and any wells with labeled HUBI complex are assayed. Data obtained using different concentrations of HUBI are used to calculate values for the number, affinity, and association of HUBI with the candidate molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An isolated cDNA which encodes a protein having the amino acid sequence of SEQ ID NO:17 and a complement of the encoding cDNA.

2. An isolated cDNA comprising a polynucleotide having a nucleic acid sequence of SEQ ID NO:18 and a complement of the polynucleotide.

3. A composition comprising the cDNA of claim 1 and a labeling moiety.

4. A substrate upon which the cDNA of claim 1 is immobilized.

5. A method for detecting differential expression in a sample containing nucleic acids, the method comprising:
a) combining the cDNA of claim 1 with the nucleic acids under conditions for formation of hybridization complexes, and
b) comparing the complexes so formed with standards, thereby establishing differential expression in the sample.

6. The method of claim 5 wherein the nucleic acids are amplified prior to hybridization.

7. The method of claim 5 wherein the sample is from lung or bladder.

8. A method of using a cDNA to screen a plurality of molecules or compounds to identify a molecule or compound which specifically binds the cDNA, the method comprising:
a) combining the cDNA of claim 1 with the plurality of molecules or compounds under conditions to allow specific binding; and
b) detecting specific binding between the cDNA and at least one molecule or compound, thereby identifying a molecule or compound that specifically binds the cDNA.

9. The method of claim 8 wherein the plurality of molecules or compounds are selected from antisense molecules, artificial chromosome constructions, branched nucleic acids, DNA molecules, enhancers, peptides, peptide nucleic acids, proteins, RNA molecules, repressors, and transcription factors.

10. A vector comprising the cDNA of claim 1.

11. A host cell comprising the vector of claim 10.

12. A method for producing a protein, the method comprising the steps of:
a) culturing the host cell of claim 11 under conditions for expression of protein; and
b) recovering the protein from the host cell culture.

13. A transgenic cell line comprising the cDNA of claim 1.

14. An antibody that specifically binds the protein produced by the method of claim 12.

15. A method for using a protein to identify an antibody comprising:
a) contacting a plurality of antibodies with the protein produce by the method of claim 12 under conditions to allow specific binding,
b) detecting specific binding between an antibody and the protein, thereby identifying an antibody that specifically binds the protein.

16. The method of claim 15, wherein the plurality of antibodies are selected from an intact immunoglobulin molecule, a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

17. A polyclonal antibody produced by the method of claim 15.

18. A monoclonal antibody produced by the method of claim 15.

19. A method for using an antibody to detect expression of a protein in a sample, the method comprising:
a) combining the antibody of claim 14 with a sample under conditions which allow the formation of antibody:protein complexes; and
b) detecting complex formation, wherein complex formation indicates expression of the protein in the sample.

20. The method of claim 19 wherein the sample is from lung or bladder.

21. The method of claim 19 wherein complex formation is compared with standards and is diagnostic of lung or bladder cancer.

22. A method for using an antibody to immunopurify a protein comprising:
a) attaching the antibody of claim 14 to a substrate,
b) exposing the antibody to a sample containing protein under conditions to allow antibody:protein complexes to form,
c) dissociating the protein from the complex, and
d) collecting the purified protein.

23. A composition comprising the antibody of claim 14 and a pharmaceutical agent.
